(19) United States
(12) Patent Application Publication
(10) Pub. No.: US 2010/0267661 A1
(43) Pub. Date: Oct. 21, 2010

(54) BETA GLUCANS AND METHODS OF USE THEREOF

(75) Inventor: Alan B. Weitberg, Newport, RI (US)

Correspondence Address: DLA PIPER LLP (US), 4365 EXECUTIVE DRIVE, SUITE 1100, SAN DIEGO, CA, 92121-2133, US

(73) Assignee: ImmuDyne, Inc., Mt. Kisco, NY (US)

(21) Appl. No.: 12/726,175

(22) Filed: Mar. 17, 2010

Related U.S. Application Data
(60) Provisional application No. 61/161,024, filed on Mar. 17, 2009.

(51) Int. Cl. A61K 31/715; A61P 35/00; A61P 7/00; A61P 35/02; C12N 5/00

(52) U.S. Cl. 514/54; 435/375

(57) ABSTRACT

The present invention relates to therapeutic uses of beta glucan for treating cancer, cytopenia, and symptoms associated with negative side effects of chemotherapy. As such, the current invention provides methods of using beta glucan for treating cancer, for increasing hematopoiesis, and for improving the quality of life of subjects undergoing chemotherapeutic treatment.

### CROSS REFERENCE TO RELATED APPLICATION(S)

This application claims the benefit of priority under 35 U.S.C. §119(e) of U.S. Ser. No. 61/161,024, filed Mar. 17, 2009, the entire content of which is incorporated herein by reference.

### BACKGROUND OF THE INVENTION

1. Field of the Invention

The invention relates generally to therapeutic uses of beta glucans and more specifically to use of beta glucans for treating cancer and side effects of chemotherapy.

2. Background Information

Cancer remains one of the most significant health problems world wide, and ranks second only to heart disease as a leading cause of death in the United States. Cancer, for the most part, involves uncontrolled proliferation and altered differentiation of the involved cells. Although the causes of most cancers are not identified and the mechanisms remain obscure, human, epidemiological, and experimental efforts have generated considerable information on the attributes of cancer. Many factors that are normally important in cell growth and differentiation in healthy individuals can contribute to the genesis or progression of the carcinogenic process in certain disease states.

Although progress is being made in understanding the biochemical and genetic mechanisms responsible for many cancers, very few successful treatment options currently exist. Unfortunately, even the most effective therapies have significant negative systemic side effects and toxicity that can be intolerable to the patient. Typical negative side effects can include, for example, nausea and vomiting, hair loss, anemia, depression of the immune system leading to infection and sepsis, and other toxic effects. Because these effects on a patient can sometimes be as debilitating as the disease being treated, the effectiveness of these current therapies is severely limited.

Beta glucans (β-glucans) are long chain polymer of glucose from the fungal cell wall which has been shown to have a number of immunomodulatory properties as well as effects on hematopoiesis and as a radiation protectant. It has been well-demonstrated that β-glucans increase neutrophil chemotaxis and adhesion, synergize with myeloid growth factors to enhance hematopoiesis and mobilize peripheral blood progenitor cells in vivo, directly stimulate committed myeloid progenitor cells, and improve survival and hematopoietic regeneration in irradiated mice. In addition, β-glucans have been shown to amplify the phagocytic killing of opsonized tumor cells and combine with monoclonal antibodies to increase their tumoricidal activity.

Accordingly, there is a need for new methods and compositions for treating cellular proliferative disorders such as cancer, and for ameliorating the negative side effects of current therapeutic approaches.

### SUMMARY OF THE INVENTION

The present invention provides formulations and methods of treating a variety of cellular proliferative disorders such as cancer. As such, the invention provides a method for treating cancer in a subject. The method includes administering to the subject a therapeutically effective amount of beta glucan. In one embodiment, the beta glucan is β-(1,3)/(1,6) D-glucan. In another embodiment, the cancer is a tumor. In another embodiment, the cancer is lymphoma, leukemia, prostate cancer, breast cancer, liver cancer, renal cancer, cervical carcinoma, colon cancer, pancreatic cancer, lung cancer, chondrosarcoma, or myelodysplastic syndrome. In another embodiment, the beta glucan is administered in combination with chemotherapy.

In another aspect, the present invention provides a method of treating cytopenia in a subject. The method includes administering to the subject a therapeutically effective amount of beta glucan. In one embodiment, the beta glucan is β-(1,3)/(1,6) D-glucan. In another embodiment, the blood cell count is increased in the subject as compared to the blood cell count prior to administration. Thus, the present invention also provides a method of increasing blood cell count (i.e., hematopoiesis) in a subject. The method includes administering to the subject a therapeutically effective amount of beta glucan.

In another aspect, the present invention provides a method of inhibiting growth of a cell. The method includes contacting the cell with beta glucan, for example β-(1,3)/(1,6) D-glucan. In one embodiment, the cell is a lymphoma cell, a leukemia cell, a melanoma cell, a prostate cancer cell, a breast cancer cell, a renal cancer cell, a liver cell, a cervical carcinoma cell, a colon cancer cell, a pancreatic cancer cell, a lung cancer cell, a chondrosarcoma cell, or a cell associated with myelodysplastic syndrome. In one embodiment, the contacting is performed in vivo. In another embodiment, the contacting is performed in vitro.

In another aspect, the present invention provides a method of identifying a cell proliferative disorder amenable to treatment with beta glucan, for example β-(1,3)/(1,6) D-glucan. The method includes detecting a decrease in cell proliferation in a sample of cells contacted with beta glucan as compared to cell proliferation in a corresponding untreated sample, thereby identifying a cell proliferative disorder amenable to treatment with beta glucan.

In another aspect, the present invention provides a method of ameliorating a side-effect of chemotherapy in a subject undergoing chemotherapy. The method includes administering to the subject a therapeutically effective amount of beta glucan, for example β-(1,3)/(1,6) D-glucan. In one embodiment, the side effect is cytopenia or fatigue. In another embodiment, the blood cell count is increased (i.e., hematopoiesis is increased) in the subject as compared to the blood cell count prior to administration.

In all aspects of the invention, the subject may be a mammal, such as a human. The beta glucan may be administered orally, topically, or systemically, and dosages may be administered once, twice or more times per day. Thus, beta glucan can be safely administered to patients with advanced malignancies receiving chemotherapy and that this adjunctive therapy may have beneficial effects on the blood counts in these patients.

### DETAILED DESCRIPTION OF THE INVENTION

The present invention is based on therapeutic uses of beta glucans for treating cancer and symptoms associated with negative side effects of chemotherapy.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “beta glucan” refers to polysaccharides that only contain glucose as structural components. Beta 1,3-D glucans are chains of D-glucose molecules, with the six-sided D-glucose rings connected at the 1 and 3 positions. However, there are many glucan linkages that can be present in a beta glucan (e.g., 1,3; 1,6; 2,3; 3,4; etc.). In addition, smaller side chains can branch off the 1,3 polysaccharide “backbone.” For example, some beta 1,3-D glucans (referred to as “β-(1,3)/(1,6) D-glucans“) contain 1,6 side-chains branching off from the longer beta-1,3 glucan backbone.

As a class of polysaccharides, β-(1,6) branched β-(1,3) glucans are composed of a main chain of glucose subunits linked together and branches linked to the main chain by a (1→6) β glycosidic linkage. Yeast β-(1,6) branched β-(1,3) glucan is composed of mostly of a main chain of glucose units linked by (1→3) beta glycosidic linkages (90% or more) with a variable number of relatively short side chains linked by β-(1→6) glycosidic linkages (10% or less); the chemical name for this glucan is poly-(1,3)-β-D-glucopyranosyl-(1,6)-β-D-glucopyranose. There are several different types of beta glucans, which vary in backbone composition, branching, type of monomers or substituents, resulting in polysaccharides that have very different physical and biological properties (Metz, Ebert, and Weicher, Chromatographia 4:345,1970; Manners et al., The structure of β-(1-3) D-glucan from yeast cell walls. Biochem. J. 135:19, 1973; U.S. Pat. No. 5,223,491, incorporated herein by reference).

Whereas all the β-1,3/1,6-D-glucans have been shown to activate the immune system of vertebrate as well as invertebrate organisms, the yeast-derived β-1,3/1,6-D-glucan is a powerful activator of macrophages, NK cells, and neutrophils. Beta glucan from yeast activates the immune system by binding to a specific receptor on the cell membrane of macrophages (Czop and Kay, Isolation and characterization of β-glucan receptors on human mononuclear phagocytes. J. Exp. Med. 173:1511-1520, 1991). The activated macrophages increase their phagocytic and bactericidal activities as well as the production of a wide range of cytokines (Burgaletta, C and Golde, D W, in Immune Modulation and control of neoplasia by adjuvant Therapy (Chirigos, M. A., ed), pp 195-200. Raven Press, NY, 1978; Sherwood et al., “Glucan stimulates production of antitumor cytolytic/cytostatic factors by macrophages,” J. Biol Resp. Mod., 6:358-381; Sherwood, et al., “Enhancement of interleukins 1, and interleukins 2 production by soluble glucan”; Browder et al., “Beneficial effects of enhanced macrophage function in the trauma patient,” Ann. Surg. 211:605-613). Enhanced function of macrophages, as well NK cells, appear responsible for a number of beneficial effects of yeast beta glucan, such as increased resistance of the host to infection by bacteria, viruses, fungi and protozoan parasites (Williams et al., “Protective effect of glucan in experimentally induced candidiasis,” J. Reticuloendot. Soc. 23:479-490, 1978; Williams and DiLuzio. “Immunopharmacological modification of experimental viral diseases by glucan,” EOS JK Immunol Immunopharmacol 5:78-82, 1985; Babineau et al. “A phase II multicenter, double blind, randomized, placebo-controlled study of three dosages of an immunomodulator (PCG-glucan) in high risk surgical patients,” Arch. Surg., 129:601-609., 1994). In addition, the enhanced function of macrophages and NK cells appears to increase the host defenses against malignant tumors (Mansell et al. “Macrophage mediated destruction of human malignant cells in vivo,” J Natl Canc. Inst. 54:571-576, 1975; Williams et al. “Chemoimmunotherapy of experimental hepatic metastasis,” Hepatology, 7:1296-1304, 1985; Ueno. “Beta-1,3-D-glucan, its immune effect and its clinical use,” Jap. J. Soc. Terminal Systemic Dis. 6:151-154, 2000).

Based on these properties, the present invention demonstrates the safety of an adjunctive treatment with beta glucans in subjects with advanced malignancies receiving chemotherapy. In addition, because the β-glucans have been shown to improve hematopoiesis in animals and because chemotherapy generally induces cytopenia in humans, the present invention sought to determine if the β-glucan exerted an effect on blood counts in patients with advanced malignancies receiving chemotherapy compared with pretreatment blood counts in patients receiving chemotherapy alone.

It has been shown that these glucans can increase neutrophil chemotaxis and adhesion in several in vivo and in vitro systems. In addition, betafectin PGG-glucan was able to synergize with myeloid growth factors in vivo to enhance hematopoietic recovery and mobilize peripheral blood progenitor cells. Treatment with glucans in mice has been shown to increase survival and improve hematopoietic regeneration after irradiation and that the glucans act directly on committed myeloid progenitors to improve hematopoiesis. As used herein, the term “hematopoiesis” refers to the formation of blood or of blood cells.

Accordingly, the present invention provides a method of ameliorating a negative side-effect of chemotherapy in a subject undergoing chemotherapy. The method includes administering to the subject beta glucan, such as, for example, β-(1,3)/(1,6) D-glucan. Current chemotherapeutic techniques have a range of side effects mainly affecting the fast-dividing cells of the body. Exemplary side effects of chemotherapy include, but are not limited to, pain, nausea and vomiting, diarrhea or constipation, malnutrition, hair loss, memory loss, depression of the immune system (hence potentially lethal infections and sepsis), weight loss or gain, hemorrhage, secondary neoplasms, cardiotoxicity, hepatotoxicity, nephrotoxicity, ototoxicity, tumor lysis syndrome, fatigue, and cytopenia (e.g., anemia, leukopenia or neutropenia, thrombocytopenia, granulocytopenia, and/or pancytopenia). Thus, in one embodiment, ameliorating a negative side-effect of chemotherapy includes the formation of blood or of blood cells in the subject, thereby treating the negative side-effect. Likewise, the present invention also provides a method of treating cytopenia in a subject by administering a beta glucan to the subject, such as β-(1,3)/(1,6) D-glucan. As used herein, the term “cytopenia” refers to a condition resulting in a decreased number of cellular elements in the blood of the subject. Thus, in one embodiment, treating cytopenia includes the formation of blood or of blood cells and/or increasing blood cell count and/or increasing hematopoiesis in the subject, thereby treating cytopenia.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the term “ameliorate” means that the clinical signs and/or the symptoms associated with cancer and/or the negative side effects of chemotherapy are lessened. The signs or symptoms to be monitored will be characteristic of a particular cancer and/or chemotherapeutic regimen and will be well known to the skilled clinician, as will the methods for monitoring the signs and conditions. As used herein, the terms “reduce” and “inhibit” are used together because it is recognized that, in some cases, a decrease can be reduced below the level of detection of a particular assay. As such, it may not always be clear whether the activity of a particular cancer is “reduced” below a level of detection of an assay, or is completely “inhibited.”

The term “administration” or “administering” is defined to include the act of providing an agent of the invention or pharmaceutical composition to the subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. Thus, the beta glucan of the invention may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

The effects of glucans on tumor cell killing has included the observation that yeast β-glucan amplifies phagocytic killing of iC3b-opsonized tumor cells, combines with monoclonal antibodies to increase tumor cell death and can increase macrophage cytotoxicity to tumor cells by increasing nitrous oxide production. Interestingly, toxicological assessment of a particulate yeast (1,3)-β-D-glucan in rats revealed no adverse or toxic effects on the animals. Thus, while the specific aim of this study was to determine the safety and side effect profile of beta glucans in subjects receiving chemotherapy for advanced malignancies and the effect of the glucan on hemtopoiesis in these subjects, the subjects were also monitored for any therapeutic effect of the glucan because the β-glucans also have immunomodulatory effects as noted above.

As such, the present invention also provides a method of treating cancer in a subject. The method includes administering to the subject a therapeutically effective amount of beta glucan, such as β-(1,3)/(1,6) D-glucan. The term “therapeutically effective amount” or “effective amount” means the amount of a compound or pharmaceutical composition that will elicit the biological or medical response of a tissue, system, animal or human that is being sought by the researcher, veterinarian, medical doctor or other clinician.

The total amount of beta glucan to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of beta glucan to treat cancer and/or one or more negative side effects of chemotherapy in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, a suitable daily dose of a compound of the invention will be that amount of the compound which is the lowest dose effective to produce a therapeutic effect. Such an effective dose will generally depend upon the factors described above. Generally, oral doses of beta glucan for a patient will range from about 0.5 to about 60 mg per day which can be administered in single or multiple doses. In one embodiment, the oral dose is about 30 mg daily, wherein a subject is given 15 mg twice per day. If desired, the effective daily dose of the active compound may be administered as two, three, four, five, six or more sub-doses administered separately at appropriate intervals throughout the day, optionally, in unit dosage forms. There may be a period of no administration followed by another regimen of administration.

Likewise, the present invention also provides a method of increasing blood cell count in a subject. The method includes administering to the subject a therapeutically effective amount of beta glucan, such as β-(1,3)/(1,6) D-glucan. A comparison of the total cell number (and/or blood cell count) prior to and during therapy indicates the efficacy of the therapy. Likewise, a comparison of the severity of the side effects of chemotherapy prior to and during beta glucan therapy indicates the efficacy of beta glucan therapy.

As used herein, the term “cancer” refers to a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, do not invade or metastasize. Included in the term “cancer” is any malignant tumor including, but not limited to, carcinoma, sarcoma and lymphoma, and any non-tumor malignant cells. Exemplary cancers that can be treated or eradicated by the methods of the invention include, but are not limited to, ovarian cancer, kidney cancer, breast cancer, prostate cancer, colon cancer, liver cancer, lung cancer, leukemia, various lymphomas, Hodgkin's disease, various melanomas, and malignant tumors of the head and neck. Thus, in one embodiment, the cancer is lymphoma, leukemia, prostate cancer, breast cancer, renal cancer, cervical carcinoma, liver cancer, colon cancer, pancreatic cancer, lung cancer, chondrosarcoma, or myelodysplastic syndrome.

In one embodiment, the beta glucan (e.g., β-(1,3)/(1,6) D-glucan) is administered in combination with a chemotherapeutic agent, an anti-inflammatory agent, antihistamines, immunomodulator, therapeutic antibody or a protein kinase inhibitor, e.g., a tyrosine kinase inhibitor, to a subject in need of such treatment. While not wanting to be limiting, chemotherapeutic agents include antimetabolites, such as methotrexate, DNA cross-linking agents, such as cisplatinkarboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, cytoxan, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, gemzar, leukovorin, leukovorin, methtrexate, nexavar, hydroxyurea, lomustine, thioguanine, alkeran (melphalan), asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like.

Classes of anticancer agents suitable for use in combination with beta glucan include, but are not limited to: 1) alkaloids, including, microtubule inhibitors (e.g., Vincristine, Vinblastine, and Vindesine, etc.), microtubule stabilizers (e.g., Paclitaxel (Taxol), and Docetaxel, Taxotere, etc.), and chromatin function inhibitors, including, topoisomerase inhibitors, such as, epipodophyllotoxins (e.g., Etoposide (VP-16), and Teniposide (VM-26), etc.), and agents that target topoisomerase I (e.g., Camptothecin and Isirinotecan (CPT-11), etc.); 2) covalent DNA-binding agents (alkylating agents), including, nitrogen mustards (e.g., Mechlorethamine, Chlorambucil, Cyclophosphamide, Ifosphamide, and Busulfan (Myleran), etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents (antitumor antibiotics), including, nucleic acid inhibitors (e.g., Dactinomycin (Actinomycin D), etc.), anthracyclines (e.g., Daunorubicin (Daunomycin, and Cerubidine), Doxorubicin (Adriamycin), and Idarubicin (Idamycin), etc.), anthracenediones (e.g., anthracycline analogues, such as, (Mitoxantrone), etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine (6-MP, Purinethol), 6-Thioguanine (6-TG), Azathioprine, Acyclovir, Ganciclovir, Chlorodeoxyadenosine, 2-Chlorodeoxyadenosine (CdA), and 2′-Deoxycoformycin (Pentostatin), etc.), pyrimidine antagonists (e.g., fluoropyrimidines (e.g., 5-fluorouracil (5 FU) (Adrucil), 5-fluorodeoxyuridine (FdUrd) (Floxuridine)) etc.), and cytosine arabinosides (e.g., Cytosar (ara-C) and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole (Arimidex), etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

In another aspect, the invention provides a method of inhibiting growth of a cell. The method includes contacting the cell with a beta glucan. In one embodiment, the beta glucan is β-(1,3)/(1,6) D-glucan. The method can be performed in vivo, for example, by administering the beta glucan to a subject having a cell undergoing uncontrolled growth (i.e., cancer). The method can be performed ex vivo, for example, by contacting a sample of cells from a subject with beta glucan and determining a change in the total cell number as a result of the contact, as compared to the total cell number of an untreated (i.e., uncontacted) cell. Detection of decreased cell number (i.e., inhibition of cell proliferation) in the contacted sample of cells as compared to the untreated cells indicates that the beta glucan inhibits growth of the cells.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, urine, and ejaculate.

Exemplary cells that can be treated by the methods of the invention include those undergoing uncontrolled cell growth as compared to corresponding normal cells. As such, exemplary cells include, but are not limited to a lymphoma cell, a leukemia cell, a melanoma cell, a prostate cancer cell, a liver cancer cell, a breast cancer cell, a renal cancer cell, a cervical carcinoma cell, a colon cancer cell, a pancreatic cancer cell, a lung cancer cell, a chondrosarcoma cell, or a cell associated with myelodysplastic syndrome.

As used herein the term “corresponding normal cells” or “corresponding normal sample” refers to cells, or a sample from a subject that is free of the cell proliferative disorder being treated and is from the same organ and of the same type as the cells being examined. In one embodiment, the corresponding normal cells comprise a sample of cells obtained from a healthy individual that does not have a cell proliferative disorder. Such corresponding normal cells can, but need not be, from an individual that is age-matched and/or of the same sex as the individual providing the cells being examined.

All methods may further include the step of bringing the active ingredient(s) (e.g., beta glucan, such as β-(1,3)/(1,6) D-glucan) into association with a pharmaceutically and/or neutraceutically acceptable carrier, which constitutes one or more accessory ingredients. As such, the invention also provides compositions for use in treating subjects having cancer and/or having one or more negative side effects of chemotherapy.

Pharmaceutically acceptable carriers useful for formulating beta glucan for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

In another aspect, the invention provides a method of identifying hyperproliferative cells (e.g., cancer cells) that are amenable to the treatments of the invention. The method can be performed, for example, by contacting a sample of cells to be treated with beta glucan (e.g., β-(1,3)/(1,6) D-glucan) and detecting a decrease in cell proliferation as a result of the contact, as compared to the cell proliferation in a corresponding normal or untreated cell sample. Detection of decreased cell proliferation and/or cell number (i.e., inhibition of cell proliferation) in the cells sample as compared to the untreated cell sample indicates that the cells can benefit from treatment. As such, the methods of the invention are useful for providing a means for practicing personalized medicine, wherein treatment is tailored to a subject based on the particular characteristics of the cancer cells and/or the negative side effects from chemotherapy in the subject.

The sample of cells examined according to the present method can be obtained from the subject to be treated, or can be cells of an established cancer cell line or known hyperproliferative cells of the same type as that of the subject In one embodiment, the established cell line can be one of a panel of such cell lines, wherein the panel can include different cell lines of the same type of disease and/or different cell lines of different diseases associated with hyperproliferation. Such a panel of cell lines can be useful, for example, to practice the present method when only a small number of cells can be obtained from the subject to be treated, thus providing a surrogate sample of the subject's cells, and also can be useful to include as control samples in practicing the present methods.

Once disease is established and a treatment protocol is initiated, the methods of the invention may be repeated on a regular basis to evaluate whether the cancer cells in the subject begin to show resistance to the therapy. The results obtained from successive assays may be used to show the efficacy of treatment over a period ranging from several days to months. Accordingly, the invention is also directed to methods for monitoring a therapeutic regimen for treating a subject having cancer and/or negative side effects from chemotherapy. A comparison of the total cell number (and/or blood cell count) prior to and during therapy indicates the efficacy of the therapy. Likewise, a comparison of the severity of the side effects of chemotherapy prior to and during beta glucan therapy indicates the efficacy of beta glucan therapy. Therefore, one skilled in the art will be able to recognize and adjust the therapeutic approach as needed.

The methods of the invention can be adapted to a high throughput format, thus allowing the examination of a plurality (i.e., 2, 3, 4, or more) of cell samples, beta glucans, and/or combinations of beta glucans and chemotherapeutic agents, which independently can be the same or different, in parallel. A high throughput format provides numerous advantages, including that various compositions can be tested on several samples of cells from a single subject, thus allowing, for example, for the identification of a particularly effective concentration of a particular ingredient of the composition to be administered to the subject, or for the identification of a particularly effective chemotherapeutic agents, or combination thereof, to be administered to the subject in combination with beta glucan. As such, a high throughput format allows, for example, control samples (positive controls and or negative controls) to be run in parallel with test samples, including, for example, samples of cells known to be effectively treated with beta glucan.

When performed in a high throughput (or ultra-high throughput) format, the methods can be performed on a solid support (e.g., a microtiter plate, a silicon wafer, or a glass slide), wherein samples to be contacted with an extract or combination thereof are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, test agents, and the like, can be dispensed to (or removed from) specified positions at desired times, and samples (or aliquots) can be monitored, for example, for decreased cell number.

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1

### β-(1,3)/(1,6) D-glucan Treatment

This example demonstrates the effectiveness of treatment with β-(1,3)/(1,6) D-glucan. Twenty patients with advanced malignancies receiving chemotherapy received the β-(1,3)/(1,6) D-glucan preparation, MacroForce plus IP6 (ImmuDyne, Mount Kisco, N.Y.), one tablet twice a day by mouth. All patients had received at least one course of chemotherapy prior to entering the study, were between 38 and 84 years old and had a performance status of 0 to 2, according to the criteria of the World Health Organization.

Patients were monitored every two weeks for side effects and complete blood counts were obtained monthly for six months. Specifically, patients were asked to report any new symptoms not experienced prior to initiation of the study and to note if any prior chemotherapy-related symptoms (e.g., nausea and vomiting) had changed after the initiation of the study. If grade 3 or 4 hematologic toxicity developed, defined as an absolute neutrophil count of less than 1000 μL or a platelet count of less than 50,000 μL, therapy would be withheld until the toxicity resolved. After resolution, dosing would be reduced to one tablet by mouth per day. Mean changes in blood counts during the study were compared to the mean blood counts observed prior to the initiation of the study. The primary endpoint was to determine the tolerability of the treatment and its effect on serial blood counts.

Table 1 demonstrates the characteristics of the patients enrolled in this study. Table 2 notes the types of malignancies experienced by these patients and the treatments they were receiving. None of the twenty patients reported any new symptoms while taking the β-glucan. Sixty per cent of the patients reported a sense of well-being while taking the β-glucan and asked to remain on the treatment even after the completion of the study. Forty per cent of the patients who experienced fatigue during their chemotherapy treatments prior to entering the study reported feeling less fatigued while taking the β-glucan. In addition, one patient with lymphoma and significant cervical adenopathy who delayed his standard chemotherapy for 4 weeks during the study and only took the β-glucan, noted a marked reduction in the size of the nodes while taking the β-glucan alone.

TABLE 1
Patient Characteristics

| | Study Patients |
|---|---|
| Characteristic | (N = 20) |
| Median age-yrs (range) | 65 (38-84) |

Sex (no.)

| | |
|---|---|
| Male | 10 |
| Female | 10 |

WHO Performance Score

| | |
|---|---|
| 0 | 14 |
| 1 | 5 |
| 2 | 1 |

TABLE 2
Patients' Diagnoses and Treatments

| Patient | | |
|---|---|---|
| # | Diagnosis | Treatment |
| 1 | non-small cell lung cancer | carboplatin/taxol |
| 2 | pancreatic carcinoma | VP-16/gemzar |
| 3 | breast carcinoma | carboplatin/taxotere |
| 4 | breast carcinoma | adriamycin/taxotere |
| 5 | non-small cell lung cancer | carboplatin/taxol |
| 6 | non-small cell lung cancer | carboplatin/taxol |
| 7 | small cell lung cancer | VP-16/cisplatin |
| 8 | colon cancer | 5 FU/leukovorin/oxaliplatin |
| 9 | breast cancer | adriamycin/taxotere |
| 10 | breast cancer | carboplatin/taxol |
| 11 | follicular lymphoma | cytoxan/vincristine/prednisone |
| 12 | renal cell carcinoma | cytoxan/nexavar |
| 13 | chondrosarcoma | cytoxan/adriamycin/ifosphamide |
| 14 | colon cancer | 5 FU/leukovorin/oxaliplatin |
| 15 | breast cancer | adriamycin/taxotere |
| 16 | follicular lymphoma | cytoxan/vincristine/prednisone |
| 17 | chronic lymphocytic leukemia | alkeran/prednisone |
| 18 | chronic lymphocytic leukemia | alkeran/prednisone |
| 19 | pancreatic carcinoma | doxorubicin/gemzar |
| 20 | myelodysplastic syndrome | hydroxyurea |

Table 3 shows the comparison of the mean blood counts prior to entering the study with the mean blood counts during the study.

TABLE 3
Change in Mean Complete Blood Count Values

| Patient | White blood | Hemoglobin | Platelet |
|---|---|---|---|
| # | count (μl) | (gm/dl) | count (μl) |
| 1 | +100 | +.03 | +26,000 |
| 2 | +1100 | +.30 | +23,000 |
| 3 | +1700 | +1.0 | +8,000 |
| 4 | +1000 | No change | −23,000 |
| 5 | No change | No change | No change |
| 6 | +1800 | −0.2 | +15,000 |
| 7 | No change | No change | No change |
| 8 | +600 | +.60 | No change |
| 9 | No change | No change | −4,000 |
| 10 | +300 | +.30 | No change |
| 11 | +700 | +.40 | −21,000 |
| 12 | No change | No change | No change |
| 13 | +300 | No change | +33,000 |
| 14 | No change | No change | No change |
| 15 | No change | No change | No change |
| 16 | +600 | +.70 | +3,000 |
| 17 | +200 | +.20 | No change |
| 18 | +100 | No change | No change |
| 19 | No change | No change | No change |
| 20 | +600 | +.05 | +5,500 |

As can be seen, in general, there was a trend to improved blood counts during the study as compared with the pre-study period. There appeared to be improvement especially of the white blood count during the study period on this dose of β-glucan. Improvements were also noted in the levels of hemoglobin and the platelet counts. Accordingly, the present invention demonstrates that β-(1,3)/(1,6) D-glucan is extremely well-tolerated in patients with advanced malignancies receiving chemotherapy. No adverse effects or toxicities were reported by any of the patients when compared to their symptom profile before entering the study. On the contrary, a significant number of patients reported a sense of well-being while taking the glucan.

There clearly was some amelioration of the blood counts in patients taking the glucan as compared to the pretreatment mean counts. This effect supports the data from animal studies which demonstrates that the beta glucans improve hematopoiesis through a variety of mechanisms. Lastly, because of the immunomodulatory properties of the glucans, these agents may have direct tumoricidal. For example one patient with lymphoma and enlarged cervical nodes noted marked improvement on the beta glucan alone.

Although the invention has been described with reference to the above example, it will be understood that modifications and variations are encompassed within the spirit and scope of the invention. Accordingly, the invention is limited only by the following claims.

What is claimed is:

1. A method of treating cancer in a subject comprising administering to the subject a therapeutically effective amount of beta glucan.

2. The method of claim 1, wherein the beta glucan is β-(1,3)/(1,6) D-glucan.

3. The method of claim 1, wherein the cancer is a tumor.

4. The method of claim 1, wherein the cancer is lymphoma, leukemia, prostate cancer, breast cancer, renal cancer, cervical carcinoma, colon cancer, pancreatic cancer, lung cancer, liver cancer, chondrosarcoma, or myelodysplastic syndrome.

5. The method of claim 1, wherein the beta glucan is administered in combination with chemotherapy.

6. The method of claim 1, wherein the beta glucan is administered twice per day.

7. The method of claim 1, wherein the beta glucan is administered orally.

8. The method of claim 1, wherein the subject is human.

9. The method of claim 1, wherein blood cell count of the subject is increased as compared to the blood cell count prior to administration.

10. A method of treating cytopenia in a subject comprising administering to the subject a beta glucan, thereby treating cytopenia.

11. The method of claim 10, wherein the beta glucan is β-(1,3)/(1,6) D-glucan.

12. The method of claim 10, wherein the beta glucan is administered twice per day.

13. The method of claim 10, wherein the beta glucan is administered orally

14. The method of claim 10, wherein the subject is human.

15. The method of claim 10, wherein blood cell count of the subject is increased as compared to the blood cell count prior to administration.

16. A method of inhibiting growth of a cell comprising contacting the cell with a beta glucan.

17. The method of claim 16, wherein the beta glucan is β-(1,3)/(1,6) D-glucan.

18. The method of claim 16, wherein the cell is selected from the group consisting of a lymphoma cell, a leukemia cell, a melanoma cell, a prostate cancer cell, a breast cancer cell, a renal cancer cell, a cervical carcinoma cell, a colon cancer cell, a pancreatic cancer cell, a liver cancer cell, a lung cancer cell, a chondrosarcoma cell, and a cell associated with myelodysplastic syndrome.

19. The method of claim 16, wherein the beta glucan is administered in combination with chemotherapy.

20. The method of claim 16, wherein the contacting is performed in vivo.

21. The method of claim 16, wherein the contacting is performed in vitro.
